(19) United States
(12) Patent Application Publication
(10) Pub. No.: US 2022/0305145 A1
(43) Pub. Date: Sep. 29, 2022

(54) COMPOSITIONS AND METHODS FOR DIAGNOSING VIRAL INFECTIONS INCLUDING COVID-19 AND FOR INFECTION SEVERITY MONITORING AS WELL AS TARGETED DETECTION OF CYTOKINE STORM

(71) Applicant: Vyripharm Enterprises, Inc., Houston, TX (US)

(72) Inventor: Jerry L. BRYANT, Jr., Bellaire, TX (US)

(21) Appl. No.: 17/697,384

(22) Filed: Mar. 17, 2022

Related U.S. Application Data
(60) Provisional application No. 63/166,904, filed on Mar. 26, 2021.

(51) Int. Cl. A61K 51/04; A61K 45/06

(52) U.S. Cl. CPC A61K 51/0482; A61K 45/06

(57) ABSTRACT

Compositions and methods for the diagnosis and treatment of infectious diseases, including for the diagnosis and treatment of Cytokine Storm and Cytokine Release Syndrome. The compositions may include a conjugate of a nucleoside analog, a chelator, and a label for use as imaging and therapeutic agents.

### CROSS-REFERENCE TO RELATED APPLICATIONS

This application claims the benefit of priority to U.S. Provisional Application Ser. No. 63/166,904, filed Mar. 26, 2021, the entire contents of which is hereby incorporated by reference, for all purposes, in its entirety.

### FIELD OF TECHNOLOGY

The present disclosure is directed to compositions and methods for targeted infectious disease diagnosis as well as compositions and methods for the treatment of infectious diseases. The present disclosure is also related to nucleoside analog chelator conjugate compositions and methods for diagnostic and prognostic applications in infectious diseases as well as for use in the treatment of infectious diseases.

### BACKGROUND

The diagnosis and treatment of many infectious diseases continues to be poorly understood. In particular, since the COVID-19 outbreak was declared a public health emergency of international concern by the World Health Organization (WHO) on Jan. 30, 2020, the progression of the severe acute respiratory syndrome coronavirus 2 (SARS-COV-2) virus has reminded us of the critical role of an effective host immune response as well as the devastating effect of immune dysregulation. Accordingly, there is a critical need for an efficient approach for both the diagnosis and treatment of infectious diseases, such as COVID-19. However, inconsistencies in the diagnosis and stage identification of infectious diseases and their progression persist making identification of appropriate and efficient treatment protocols challenging. In particular, the diagnosis and prognosis of Cytokine Storm and Cytokine Release Syndrome associated with infectious disease progression has proved difficult. Cytokine Storm and Cytokine Release Syndrome are life-threatening systemic inflammatory syndromes involving elevated levels of circulating cytokines and immune-cell hyperactivation that can be triggered by various therapies, pathogens, autoimmune conditions, and monogenic disorders. Furthermore, effective approaches to tailored or targeted treatment of infectious diseases has also been challenging, including attempts at developing vaccines. Accordingly, there is a need for improved approaches for the accurate diagnosis as well as evaluation of disease progression and severity in order to provide patient-specific targeted treatment protocols and methodologies. There is also a need for improved methods of evaluating patient-specific treatment outcomes and for the effective diagnosis and forecasting of Cytokine Storm and Cytokine Release Syndrome, as well as other symptoms and conditions related to infectious disease progression.

### BRIEF DESCRIPTION OF THE DRAWINGS

In order to describe the manner in which the advantages and features of the disclosure can be obtained, reference is made to embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1 is exemplary process for the synthesis of the compound according to structural formula I, N-(4-(2-amino-6-oxo-1,6,-dihydro-9H-purin-9-yl)-2-(hydroxymethyl)butyl)-2-(1,4,8,11-tetraazacyclotetradecan-1-yl)acetamide, according to an exemplary embodiment of the present disclosure; and

FIG. 2 is an exemplary process for the synthesis of the compound according to structural formula IV, 1,4,8,11-tetraazacyclotetradecane-1′-acetyl-[N-(Piperidin-1-yl)-5 -(4-chlorophenyl)-1-(2,4-dichlorophenyl)-4-methyl-1H-pyrazole-3-carboxamide], according to an exemplary embodiment of the present disclosure.

### DETAILED DESCRIPTION

It will be appreciated that numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein.

The present disclosure provides compositions and methods for diagnosing and/or treating infectious diseases. In some instances, the compositions may include a conjugate of a nucleoside and chelator. In other cases, the compositions may include a conjugate of a nucleoside analog, a chelator, and a label. The presently disclosed compositions may be used to evaluate outcomes of an infectious disease or symptoms and disorders related to infectious disease infections. The presently disclosed compositions and methods using the compositions are amenable to providing personalized treatment that is critical to the effective treatment of infectious diseases in individuals that may respond differently to the infectious agents and various treatment protocols. The presently disclosed methods and compositions are safe and cost-effective and the diagnostic methods are non-invasive.

The nucleoside analog may be a guanine analog. In other cases, the nucleoside analog may be a cell replication check point ligand. In some instances, the nucleoside analog may be a synthetic analog. In other instances, the nucleoside analog may be a natural analog. In some cases, the nucleoside analog may be guanine. According to at least one aspect, the nucleoside analog may be selected from the group consisting of adenine, adenosine, deoxyadenosine, guanine, guanosine, dexoyguanosine, thymine, 5-methyluridine, thymidine, uracile, uridine, deoxyuridine, cytosine, cytidine, deoxycytidine, and any combination thereof. The nucleoside analog may, in some instances, be arabinosyl nucleoside.

In some instances, the chelator may be an aminated chelator or an acid chelator. In some instances, the chelator may be a N4 chelator or ligand. The chelator, may be, for example, cyclam, 6-carboxy-1,4,8,11-tetraazaundecane, or 1,4,8,11-tetraazabicyclohexadecane.

The label may be a radionuclide label. The radionuclide label may be selected from the group consisting of Technetium-99, Gallium-68, Copper-60, Copper-64, Indium-111, Holmium-166, Rhenium-186, Rhenium -188, Yttrium-90, Lutetium-177, Radium-223, Actinium-225, and any combination thereof. In at least some instances, the radionuclide label may be configured to facilitate contrast-enhanced imaging when administered to a mammalian subject in conjunction with diagnostic imaging.

The conjugate composition may be a N4-guanine (N4amG) such as cyclam-amguanine. In some instances, the conjugate may comprise N-(4-(2-amino-6-oxo-1,6,-dihydro-9H-purin-9-yl)-2-(hydroxymethyl)butyl)-2-(1,4,8,11-tetraazacyclotetradecan-1-yl)acetamide, corresponding to a compound characterized by the structure according to Formula I:

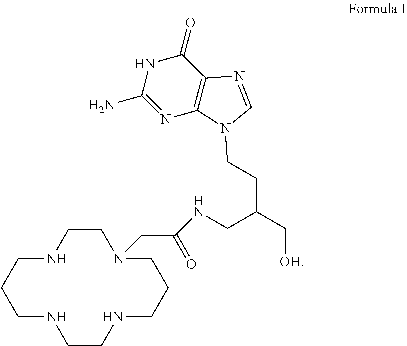 [SMILES: Nc1nc2c(ncn2CCC(CO)CNC(=O)CN2CCCNCCNCCCNCC2)c(=O)[nH]1]

In other instances, the conjugate may comprise N-(9-(4-amino-3-(hydroxymethyl)butyl)-6-oxo-6,9-dihydro-1H-purin-2-yl)-2-(1,4,8,11-tetraazacyclotetradecan-1-yl)acetamide, corresponding to a compound characterized by the structure according to Formula II:

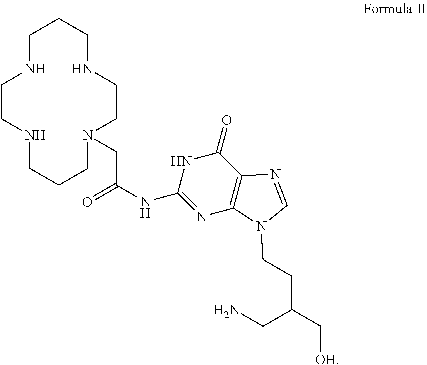 [SMILES: NCC(CO)CCn1cnc2c(=O)[nH]c(NC(=O)CN3CCCNCCNCCCNCC3)nc21]

In still other instances, the conjugate may comprise N-(9-(4-(2-(1,4,8,11-tetraazacyclotetradecan-1-yl)acetamido-3-(hydroxymethyl)butyl)-6-oxo-6,9-dihydro-1H-purin-2-yl)-2-(1,4,8,11-tetraazacyclotetradecan-1-yl)acetamide, corresponding to a compound characterized by the structure according to Formula III:

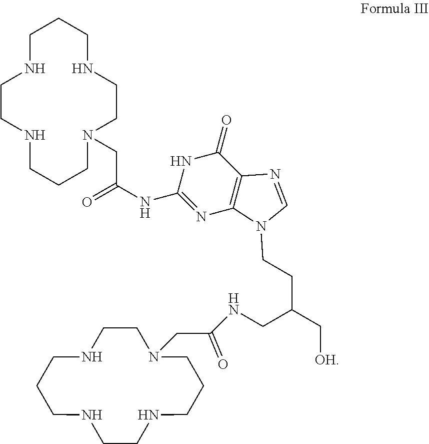 [SMILES: O=C(CN1CCCNCCNCCCNCC1)NCC(CO)CCn1cnc2c(=O)[nH]c(NC(=O)CN3CCCNCCNCCCNCC3)nc21]

In still other instances, the conjugate may comprise 1,4,8,11-tetraazacyclotetradecane-1′-acetyl-[N-(Piperidin-1-yl)-5 -(4-chlorophenyl)-1-(2,4-dichlorophenyl)-4-methyl-1H-pyrazole-3-carboxamide], corresponding to a compound characterized by the structure according to Formula IV:

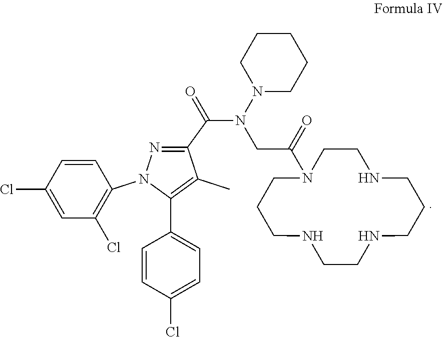 [SMILES: Cc1c(C(=O)N(CC(=O)N2CCCNCCNCCCNCC2)N2CCCCC2)nn(-c2ccc(Cl)cc2Cl)c1-c1ccc(Cl)cc1]

Any of the conjugates disclosed herein, such as the conjugates according to Formula I, Formula II, Formula III, Formula IV, may further be labeled with a radionuclide label to form the compositions comprising a conjugate of a nucleoside analog, a chelator, and a label. For example, the conjugates in Formulas I-IV may include a label selected from the group consisting of Technetium-99, Gallium-68, Copper-60, Copper-64, Indium-111, Holmium-166, Rhenium-186, Rhenium -188, Yttrium-90, Lutetium-177, Radium-223, Actinium-225, and any combination thereof.

The presently disclosed compositions may be used in various methods for diagnosing or treating infectious diseases. In particular, the present disclosure provides methods for diagnosing an infectious disease in a subject in need thereof. The method may include administering to the subject a pharmaceutically effective amount of one or more of the presently disclosed compositions. The method may also include performing an imaging technique on the subject or a portion thereof which is capable of detecting one or more signals from the composition.

The present disclosure also provides methods for determining the stage of progression of an infectious disease in a subject in need thereof. The methods may include administering to the subject a pharmaceutically effective amount of one or more of the presently disclosed compositions. The methods may also include performing an imaging technique on the subject or a portion thereof which is capable of detecting one or more signals from the composition.

The present disclosure also provides methods for diagnosing or forecasting Cytokine Storm or Cytokine Release Syndrome in a subject having an infectious disease. The methods may include administering to the subject a pharmaceutically effective amount of one or more of the presently disclosed compositions. The method may also include performing an imaging technique on the subject or a portion thereof which is capable of detecting one or more signals from the composition.

The present disclosure also provides methods for monitoring an infectious disease in a subject in need thereof. The methods may include administering to the subject a pharmaceutically effective amount of one or more of the presently disclosed compositions. The method may also include performing an imaging technique on the subject or a portion thereof which is capable of detecting one or more signals from the composition.

The present disclosure also provides methods for imaging a subject having an infectious disease in a subject. The methods may include administering to the subject a pharmaceutically effective amount of one or more of the presently disclosed compositions. The method may also include performing an imaging technique on the subject or a portion thereof which is capable of detecting one or more signals from the composition.

The present disclosure also provides methods for imaging a plurality of cells in a subject wherein the plurality of cells are infected with an infectious disease pathogen. The methods may include administering to the subject a pharmaceutically effective amount of one or more of the presently disclosed compositions in a manner such that the plurality of cells effectively receive the composition. The method may also include performing an imaging technique on at least a portion of the subject containing the plurality of cells which is capable of detecting one or more signals from the composition.

The present disclosure also provides methods for treating an infectious disease in a subject in need thereof. The methods may include administering to the subject a pharmaceutically effective amount of one or more of the presently disclosed compositions. The method may also include performing an imaging technique on the subject or a portion thereof which is capable of detecting one or more signals from the composition. The method may also include making at least one treatment decision based on the results of the imaging technique performed on the subject.

The present disclosure also provides methods for treating or preventing Cytokine Storm of Cytokine Release Syndrome in a subject having an infectious disease. The methods may include administering to the subject a pharmaceutically effective amount of one or more of the presently disclosed compositions. The method may also include performing an imaging technique on the subject or a portion thereof which is capable of detecting one or more signals from the composition. The method may also include making at least one treatment decision based on the results of the imaging technique performed on the subject.

The imaging technique may be any imaging technique capable of detecting one or more signals from the image probe composition. For example, the imaging technique may be selected from the group consisting of positron emission tomography (PET), computed tomography (CT), single photon emission computed tomography (SPECT), magnetic resonance imaging (MRI), near-infrared (NIR), optical imaging, optoacoustic imaging, ultrasound, and any combination thereof

In at least some instances, the infectious disease is a viral infection. For instance, the infectious disease may be a respiratory viral infection selected from the group consisting of human influenza, the common cold, Middle East respiratory syndrome (MERS), severe acute respiratory syndrome coronavirus (SARS), and COVID-19. The infectious disease may also be caused by infection by a virus selected from the group consisting of severe acute respiratory syndrome coronavirus (SARS-CoV), severe acute respiratory syndrome coronavirus 2 (SARS-CoV-2), Middle East respiratory syndrome-related coronavirus (MERS-CoV), human coronavirus NL63 (HCoV NL63), human coronavirus OC43 (HCoV-OCC43), human coronavirus HKU1 (HCoV HKU1), and human coronavirus 229E (HCoV-229E).

As used herein, the term “conjugate,” in all its forms, refers to a compound formed by the joining of two or more chemical compounds. The term “pharmaceutically acceptable derivative,” as used herein, refers to and includes any pharmaceutically acceptable salt, pro-drug, metabolite, ester, ether, hydrate, polymorph, solvate, complex, and adduct of a compound described herein, which, upon administration to a subject, is capable of providing (directly or indirectly) the active ingredient. For example, the term “a pharmaceutically acceptable derivative” of compounds described herein includes all derivatives of the compounds described herein (such as salts, pro-drugs, metabolites, esters, ethers, hydrates, polymorphs, solvates, complexes, and adducts) which, upon administration to a subject, are capable of providing (directly or indirectly) the compounds described herein. As used herein, the term “pharmaceutically acceptable salt” refers to those salts, which retain the biological effectiveness and properties of the parent compound. Unless otherwise indicated, a pharmaceutically acceptable salt includes salts of acidic or basic groups, which may be present in the compounds of the formulae disclosed herein. The present disclosure also provides certain processes, as examples, for the preparation of the above pharmaceutically acceptable salts, their derivatives, their analogs, their tautomeric forms, their stereoisomers, their polymorphs, and pharmaceutical compositions containing them.

Certain embodiments of the present disclosure relate to pharmaceutically acceptable salts formed by the compounds described herein, or their derivatives, their analogs, their tautomeric forms, their stereoisomers, their polymorphs and pharmaceutically acceptable compositions containing them. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids, phenylsubstituted alkanoic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, beta-hydroxybutyrate, chloride, cinnamate, citrate, formate, fumarate, glycolate, heptanoate, lactate, maleate, hydroxymaleate, malonate, mesylate, nitrate, oxalate, phthalate, phosphate, monohydro genphosphate, dihydrogenphosphate, metaphosphate, pyrophosphate, propionate, phenylpropionate, salicylate, succinate, sulfate, bisulfate, pyrosulfate, sulfite, bisulfate, sulfonate, benzenesulfonate, p-bromophenylsulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like.

Combinations containing the label-chelator-nucleoside analog conjugates combine imaging with the therapeutic intervention and can image in real time the uptake and activity of N4 conjugated nucleoside analog, which is essential to select the individual patient with the targeted dysfunctional pathway (right disease) and to assess optimal dosage (right dose). This approach allows for visually seeing the composition located at the tissue site and determining the actual dose of uptake to that site for that patient. This platform allows one to evaluate: (a) if dosing is the cause of the adverse event; (b) if bioavailability is the cause or (c) if there is a limited uptake and/or bio-distribution. In keeping with these parameters, the embodiments serve to dissect effects that are patient dependent, particularly if one of these effects are genetic, epigenetic, or exhibit allelic variations associated to the individual's ECS.

The effective amount of a compound is determined based on several factors, such as age and weight of the patient, severity of the disease, other co-existing factors. The effective amount of a compound includes exemplary dosage amounts for an adult human of from about 0.1 to 100 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition.

The following descriptions of methods, compositions, and results obtained using them are provided merely as illustrative examples. Descriptions of the methods are not intended to require or imply that the steps of the various embodiments must be performed in the order presented. The steps in the foregoing embodiments may be performed in any order. Words such as “then” are not intended to limit the order of the steps; these words are simply used to guide the reader through the description of the methods. Many of the operations may be performed in parallel or concurrently. In addition, the order of the operations may be re-arranged. Various modifications to these embodiments will be readily apparent based on the description provided here, and the generic principles defined here may be applied to other embodiments without departing from the scope of the disclosure.

Further modifications and alternative embodiments of various aspects of the compositions and methods disclosed here will be apparent in view of this description. Accordingly, this description is to be construed as illustrative only and is for the purpose of teaching those skilled in the art the general manner of carrying out the embodiments. It is to be understood that the forms of the embodiments shown and described here are to be taken as examples of embodiments. Elements and materials may be substituted for those illustrated and described here, parts and processes may be reversed or omitted, and certain features of the embodiments may be utilized independently, all as would be apparent after having the benefit of this description of the embodiments. Changes may be made in the elements described here without departing from the scope of the embodiments as described in the following claims.

### EXAMPLES

### Example 1—Synthesis of the Compound According to Formula I, N-(4-(2-amino-6-oxo-1,6,-dihydro-911-purin-9-yl)-2-(hydroxymethyl)butyl)-2-(1,4,8,11-tetraazacyclotetradecan-1-yl)acetamide

The guanine nucleoside analog compound according to Formula I,

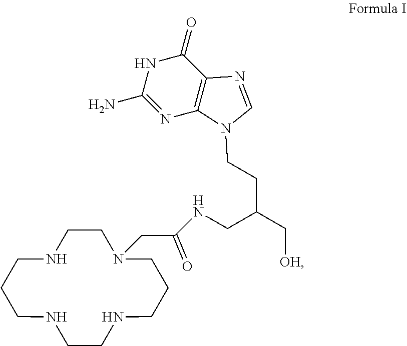 [SMILES: Nc1nc2c(ncn2CCC(CO)CNC(=O)CN2CCCNCCNCCCNCC2)c(=O)[nH]1]

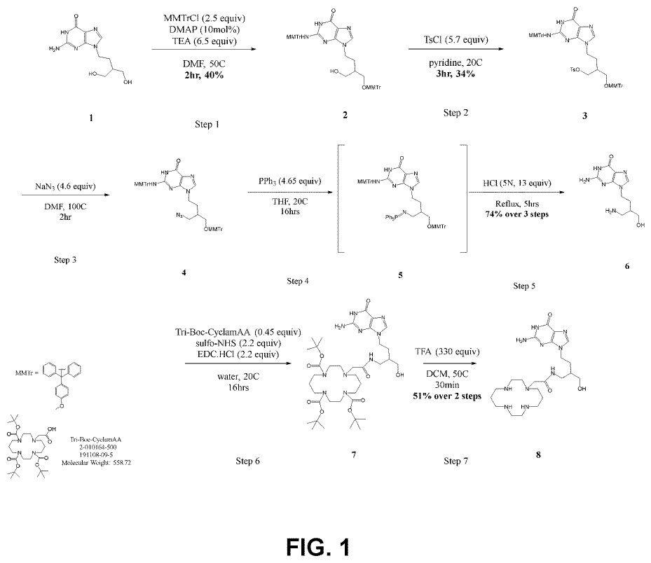

may be synthesized in several ways. FIG. 1 depicts an example process for the synthesis of the compound according to Formula I, N-(4-(2-amino-6-oxo-1,6,-dihydro-9H-purin-9-yl)-2-(hydroxymethyl)butyl)-2-(1,4,8,11-tetraazacyclotetradecan-1-yl)acetamide. As shown in FIG. 1, Compound 1 (penciclovir, 50.0 g, 1 Eq, 197 mmol) may be charged in a 1L flask with a mechanical stirrer, thermocouple, and nitrogen inlet, followed by the addition of DMSO (300 mL, 60093) (dried over 4A MS) and followed by the addition of triethylamine (44.0 g, 60.5 mL, 2.2 Eq, 434 mmol).

The mixture may be stirred for 10 min to give a white suspension. The stirring may then be increased to vigorous (400-500RPM). MMTrCl (122 g, 2.0 Eq, 395 mmol) may then be added as a solid while maintaining temperature at 20-25° C. over 20 minutes. An ice bath is then used to periodically lower the reaction temperature. Following the addition, the reaction mixture is a thick brown-black solution. After 4 hrs, the reaction mixture is poured into a mixture of 1.5L DCM and 1L water. The reaction mixture is then stirred for 5-10 minutes and let settle for 1 hr before separating the layers. The organic layer is diluted with 1L water, stirred for 5-10 minutes and let settle for 1 hr. After l1r, the mixture is filtered and the solids are discarded. The layers are separated and the organic layer is diluted with 1L water, stirred for 5-10 minutes and let settle for 1 hr. The layers are separated and after 18 hr aging, the organic layer is filtered and the solids discarded. The organic layer is then dried over sodium sulfate (75 g) and filtered and evaporated filtrate on rotavap (50 mBar, 35 C). Dried briefly under direct vacuum to give 150 g crude solids. Purified by flash chromatography on a 1.5 kg Biotage SNAP Ultra (25 uM) cartridge. The resulting compound was analyzed by proton nuclear magnetic resonance (¹H NMR), carbon-13 nuclear magnetic resonance (¹³C NMR), and high-resolution mass spectrometry (HRMS) by electrospray ionization (ESI).

Compound 2 (450 mg, 1 Eq, 564 μmol) was dissolved in Pyridine (7.5 mL) in a reaction vial with stir bar, thermocouple. Then p-toluenesulfonyl chloride (613 mg, 5.7 Eq, 3.21 mmol) was added over 10 min (9:40 AM-9:50 AM). Color darkens somewhat, very mild exotherm. Temperature remains between 20-22C. After 3.5 hrs, diluted reaction mixture with EtOAc (20 mL) and water (10 mL). Wash organic with a further 2×10mL water. Dried organic layer over Na2SO4 (500 mg-1g), evaporated to dryness. Azeotroped 2×10 mL toluene. Then azeodry 1×10 mL MeCN to yield a yellow solid. Silica chromatography (14×) using 10 g cartridge, Biotage SNAP ultra. Reaction/column monitoring at 254 nm with lambda all detection. solvent. Dissolve in 1 mL EtOAc, liquid loading. Rinse 2 mL 65% EtOAc/heptane. MPA: hept. MPB: EtOAc. The resulting compound was analyzed by proton nuclear magnetic resonance (¹H NMR), carbon-13 nuclear magnetic resonance (¹³C NMR), and high-resolution mass spectrometry (HRMS) by electrospray ionization (ESI).

In a 1L RBF with stir bar, thermocouple, nitrogen inlet dissolved Compound 3 (53.7 g, 1 Eq) in anhydrous DMF (537 mL, stored over 4A MS) then added sodium azide (5.13 g, 1.4 Eq). Heated to 50 C. After heating for 24 hrs, cooled reaction to room temperature. partitioned mixture between EtOAc (1.5L) and water (1.5L). Let settle 1hr, then split layers. Wash organic 2×1.5L water further, allowing mixture to settle for 1hr each time and discarding the rag layer. Dried organic layer over sodium sulfate (57 g). Evaporated on rotavap (35 C, 50 mBar) and dried briefly under direct vacuum to give Compound 4 as a white semisolid, 38.9 g, 75% yield. The resulting compound was analyzed by proton nuclear magnetic resonance (¹H NMR), carbon-13 nuclear magnetic resonance (¹³C NMR), and high-resolution mass spectrometry (HRMS) by electrospray ionization (ESI).

In a 50 mL RBF with stir bar, condenser, thermocouple, heating mantle, charge Compound 4 (1.00 g, 1 Eq) then THF (15 mL) and water (1.5 mL). Triphenylphosphine (344 mg, 1.2 Eq) was added, and the mixture was heated to 65 C. After 4hrs, cool reaction to 25 C, add hydrochloric acid (216 mg, 0.18 mL, 2 Eq). The mixture was heated to 65 C. After 3 hrs, cool to room temperature and filter thru 0.2 uM frit. Separated colorless lower layer, transferred to RBF and evaporated to white residue. Dried in vacuum oven (20 C, −29 inHg) overnight to give Compound 6 as a white solid (351 mg). A qNMR experiment indicates that the material is 62% potent, with the remainder of mass being water (78% adjusted yield). The resulting compound was analyzed by proton nuclear magnetic resonance (¹ H NMR), carbon-13 nuclear magnetic resonance (¹³C NMR), and high-resolution mass spectrometry (HRMS) by electrospray ionization (ESI).

In a 100 mL RBF, dilute Compound 6 aqueous solution (35.91 g, 29.8 wt %) with 24 g water. Basify to pH 8.1 with 4M NaOH. After 1.5 hrs, add 2 g of celite and filter suspension. Dry solids overnight at ambient temperature (−29 inHg) to give Compound 6 as a white solid. Solids were dissolved in 550 mL 20% DMSO/MeOH and filtered. Filtrate was evaporated on a rotavap (40 C, 50 mBar) and then under direct vacuum to give Compound 6 solution in DMSO (41.61 g, 12.6 wt %). Compound 6 DMSO solution (41.61g, 12.6 wt %) was further diluted with anhydrous DMSO (73mL) and then anhydrous DMF (212 mL). Added stir bar, nitrogen inlet, thermocouple. Added TriBocCyclamAA (12.9 g, 1.1 Eq) then DMAP (5.13 g, 2.0 Eq) and stirred until mostly dissolved. Then, added EDC.HCl (8.1g, 2.0 Eq) in a single portion at 20 C. After 24 hrs, the reaction mixture was diluted with DCM (815 mL), 160 mL water, and 650 mL sat. sodium sulfate. The pH of the aqueous layer was adjusted from 8 to 4 using 6M HCl (˜4.5 mL). The biphase was allowed to settle for 1 hr, then the layers were separated. The organic was washed 4x further with 160 mL water, 650 mL sat. sodium sulfate, maintaining the aqueous pH between 4-5 using 6M HCl. The organic layer was dried over sodium sulfate (27 g) and filtered. The sodium sulfate cake was rinsed with 150 mL DCM and the filtrate evaporated on a rotavap (50 mBar, 40 C) then under direct vacuum (−29inHg) to give 31.34 g pale yellow oil (49.1 wt %, 15.4 g intermediate 6, 92% yield). The resulting compound was analyzed by proton nuclear magnetic resonance (¹H NMR), carbon-13 nuclear magnetic resonance (¹³C NMR), and high-resolution mass spectrometry (HRMS) by electrospray ionization (ESI).

As further shown in FIG. 1, Compound 7 is dissolved (13.2 g, 1 Eq) in DCM (190 mL) and MeCN (20 mL) in 1L flask with stir bar. Add triethylsilane (19.9 mL, 7.5 Eq) then cool to 0 C. Add trifluoroacetic acid (51.3 mL, 40 Eq) maintaining temperature <10 C. Following addition warm to room temperature. After 23 hr, charge additional trifluoroacetic acid (12.5 mL, 10 Eq). After 24 hrs, dilute mixture with 40 mL water, stir for 1.5 hr, then let settle for 15 min. Collect faint purple, hazy aqueous lower layer into 1L RBF. Extract organic additional portion 40 mL water, combine colorless upper aqueous layer with previous aqueous extract. Adjust pH to 8.4 with 4M NaOH, maintaining temperature <35 C. Strip on rotavap (40 C, 50 mBar) then freeze dry overnight to give 180 g crude as an aqueous solution. The resulting compound was analyzed by proton nuclear magnetic resonance (¹H NMR), carbon-13 nuclear magnetic resonance (¹³C NMR), and high-resolution mass spectrometry (HRMS) by electrospray ionization (ESI).

### Example 2—Synthesis of the Compound According to Formula II, N-(9-(4-amino-3-(hydroxymethyl)butyl)-6-oxo-6,9-dihydro-1H-purin-2-yl)-2-(1,4,8,11-tetraazacyclotetradecan-1-yl)acetamide

Synthesis affords the constitutional isomer nucleoside analog compound according to Formula II,

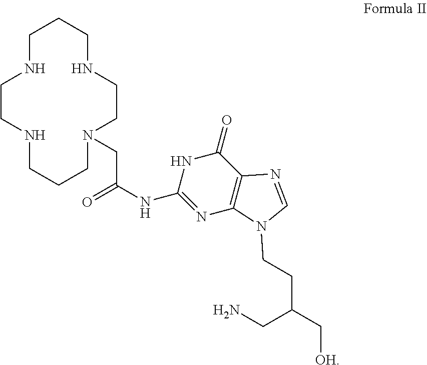 [SMILES: NCC(CO)CCn1cnc2c(=O)[nH]c(NC(=O)CN3CCCNCCNCCCNCC3)nc21]

### Example 3—Synthesis of the Compound According to Formula III, N-(9-(4-(2-(1,4,8,11-tetraazacyclotetradecan-1-yl)acetamido-3-(hydroxymethyl)butyl)-6-oxo-6,9-dihydro-1H-purin-2-yl)-2-(1,4,8,11-tetraazacyclotetradecan-1-yl)acetamide

Synthesis affords the dicyclam product nucleoside analog compound according to Formula III,

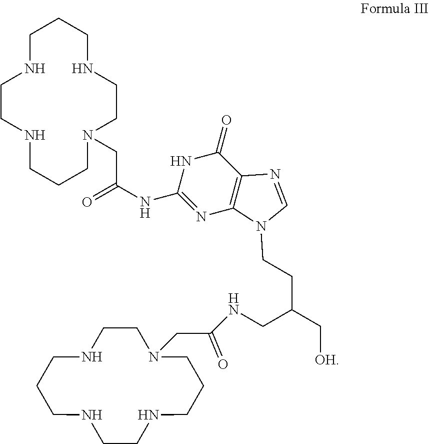 [SMILES: O=C(CN1CCCNCCNCCCNCC1)NCC(CO)CCn1cnc2c(=O)[nH]c(NC(=O)CN3CCCNCCNCCCNCC3)nc21]

### Example 4—Synthesis of the Compound According to Formula IV, 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide]

The nucleoside analog compound according to Formula IV,

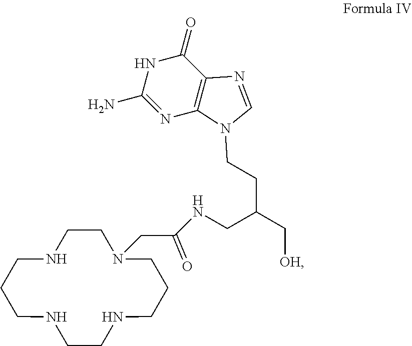 [SMILES: Nc1nc2c(ncn2CCC(CO)CNC(=O)CN2CCCNCCNCCCNCC2)c(=O)[nH]1]

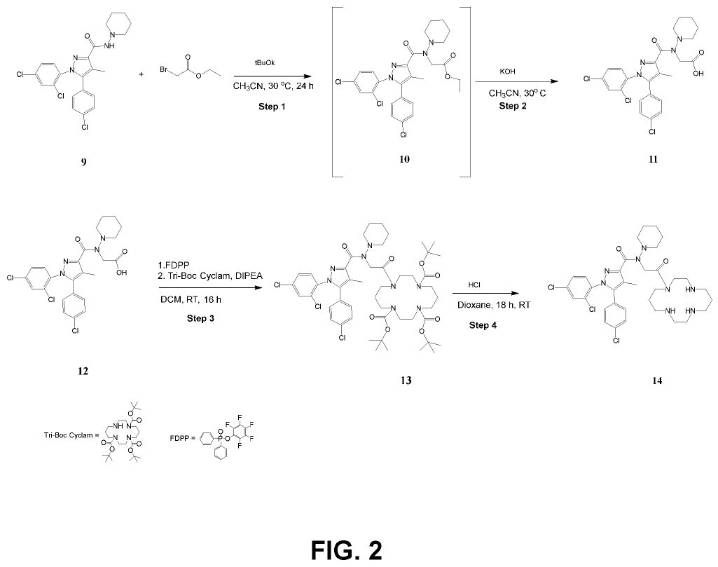

may be synthesized in several ways. FIG. 2 depicts an example process for the synthesis of the compound according to Formula IV, 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide].

Statements of the Disclosure Include:

Statement 1: A composition comprising: a conjugate of a nucleoside analog and a chelator.

Statement 2: The composition according to Statement 1, wherein the conjugate comprises a conjugate of a nucleoside analog, a chelator, and a label.

Statement 3: The composition according to Statement 1 or Statement 2, wherein the nucleoside analog is a guanine analog.

Statement 4: The composition according to Statement 1 or Statement 2, wherein the nucleoside analog is a cell replication check point ligand.

Statement 5: The composition according to Statement 1 or Statement 2, wherein the nucleoside analog is a synthetic analog.

Statement 6: The composition according to Statement 1 or Statement 2, wherein the nucleoside analog is a natural analog.

Statement 7: The composition according to Statement 1 or Statement 2, wherein the nucleoside analog is guanine.

Statement 8: The composition according to Statement 1 or Statement 2, wherein the nucleoside analog is selected from the group consisting of adenine, adenosine, deoxyadenosine, guanine, guanosine, dexoyguanosine, thymine, 5-methyluridine, thymidine, uracile, uridine, deoxyuridine, cytosine, cytidine, deoxycytidine, and any combination thereof.

Statement 9: The composition according to Statement 1 or Statement 2, wherein the nucleoside analog is arabinosyl nucleoside.

Statement 10: The composition according to any one of Statements 1-9, wherein the chelator is an aminated chelator.

Statement 11: The composition according to any one of Statements 1-9, wherein the chelator is an acid chelator.

Statement 12: The composition according to any one of Statements 1-9, wherein the chelator is cyclam.

Statement 13: The composition according to any one of Statements 1-9, wherein the chelator is a N4 chelator or ligand.

Statement 14: The composition according to any one of Statements 1-9, wherein the chelator is 6-carboxy-1,4,8,11-tetraazaundecane.

Statement 15: The composition according to any one of Statements 1-9, wherein the chelator is 1,4,8,11-tetraazabicyclohexadecane.

Statement 16: The composition according to any one of Statements 1-15, wherein the label is a radiotracer label.

Statement 17: The composition according to any one of Statements 1-15, wherein the label is a radionuclide.

Statement 18: The composition according to any one of Statements 1-15, wherein the label is a radionuclide metal ion.

Statement 19: The composition according to any one of Statements 1-15, wherein the label is selected from the group consisting of Technetium-99, Gallium-68, Copper-60, Copper-64, Indium-111, Holmium-166, Rhenium-186, Rhenium-188, Yttrium-90, Lutetium-177, Radium-223, Actinium-225, and any combination thereof.

Statement 20: The composition according to any one of Statements 1-15, wherein the label is configured to facilitate contrast-enhanced imaging when administered to a mammalian subject in conjunction with diagnostic imaging.

Statement 21: The composition according to any one of Statements 1-20, wherein the conjugate comprises N4-guanine (N4amG).

Statement 22: The composition according to any one of Statements 1-20, wherein the conjugate comprises cyclam-am-guanine.

Statement 23: The composition according to any one of Statements 1-20, wherein the conjugate comprises N-(4-(2-amino-6-oxo-1,6,-dihydro-9H-purin-9-yl)-2-(hydroxymethyl)butyl)-2-(1,4,8,11-tetraazcyclotetradecan-1-yl)acetamide.

Statement 24: The composition according to any one of Statements 1-20, wherein the conjugate comprises a conjugate compound having a structure according to Formula I:

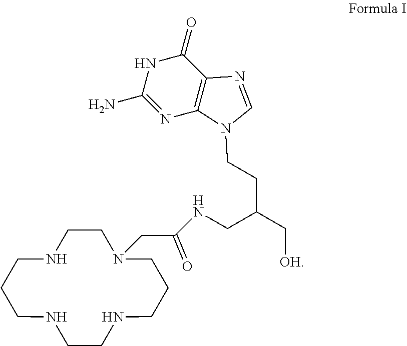 [SMILES: Nc1nc2c(ncn2CCC(CO)CNC(=O)CN2CCCNCCNCCCNCC2)c(=O)[nH]1]

Statement 25: The composition according to any one of Statements 1-20, wherein the conjugate comprises N-(9-(4-amino-3-(hydroxymethyl)butyl)-6-oxo-6,9-dihydro-1H-purin-2-yl)-2-(1,4,8,11-tetraazacyclotetradecan-1-yl)acetamide.

Statement 26: The composition according to any one of Statements 1-20, wherein the conjugate comprises a conjugate compound having a structure according to Formula II:

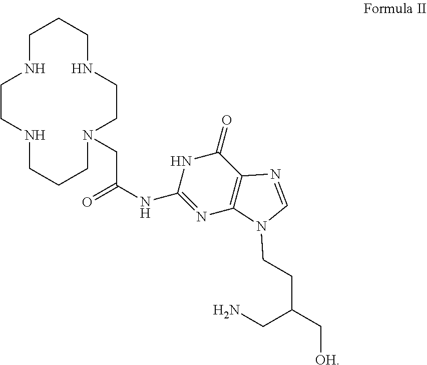 [SMILES: NCC(CO)CCn1cnc2c(=O)[nH]c(NC(=O)CN3CCCNCCNCCCNCC3)nc21]

Statement 27: The composition according to any one of Statements 1-20, wherein the conjugate comprises N-(9-(4-(2-(1,4,8,11-tetraazacyclotetradecan-1-yl)acetamido-3-(hydroxymethyl)butyl)-6-oxo-6,9-dihydro-1H-purin-2-yl)-2-(1,4,8,11-tetraazacyclotetradecan-1-yl)acetamide .

Statement 28: The composition according to any one of Statements 1-20, wherein the conjugate comprises a conjugate compound having a structure according to Formula III:

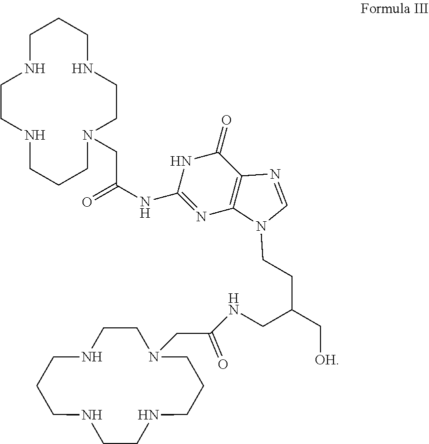 [SMILES: O=C(CN1CCCNCCNCCCNCC1)NCC(CO)CCn1cnc2c(=O)[nH]c(NC(=O)CN3CCCNCCNCCCNCC3)nc21]

Statement 29: The composition according to any one of Statements 1-20, wherein the conjugate comprises 1,4,8,11-tetraazacyclotetradecane-1′-acetyl-1′-[N-(Piperidin-1-yl)-5-(4-chlorophenyl)-1-(2,4-dichlrophenyl)-4-methyl-1H-pyrazole-3 -carboxamide].

Statement 30: The composition according to any one of Statements 1-20, wherein the conjugate comprises a conjugate compound having a structure according to Formula IV:

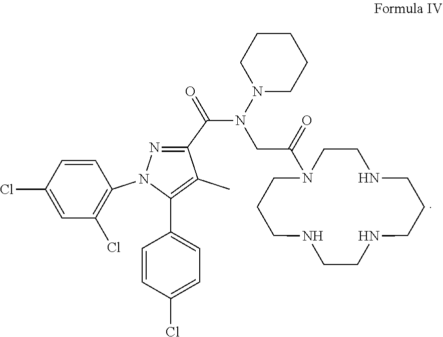 [SMILES: Cc1c(C(=O)N(CC(=O)N2CCCNCCNCCCNCC2)N2CCCCC2)nn(-c2ccc(Cl)cc2Cl)c1-c1ccc(Cl)cc1]

Statement 31: A method of diagnosing an infectious disease in a subject in need thereof, the method comprising: administering to the subject a pharmaceutically effective amount of a composition according to any one of Statements 1-30; and performing an imaging technique on the subject or a portion thereof, wherein the imaging technique is capable of detecting one or more signals from the composition.

Statement 32: A method of determining the stage of progression of an infectious disease in a subject in need thereof, the method comprising: administering to the subject a pharmaceutically effective amount of a composition according to any one of Statements 1-30; and performing an imaging technique on the subject or a portion thereof, wherein the imaging technique is capable of detecting one or more signals from the composition.

Statement 33: A method of diagnosing or forecasting Cytokine Storm or Cytokine Release Syndrome in a subject having an infectious disease, the method comprising: administering to the subject a pharmaceutically effective amount of a composition according to any one of Statements 1-30; and performing an imaging technique on the subject or a portion thereof, wherein the imaging technique is capable of detecting one or more signals from the composition.

Statement 34: A method of monitoring an infectious disease in a subject in need thereof, the method comprising: administering to the subject a pharmaceutically effective amount of a composition according to any one of Statements 1-30; and performing an imaging technique on the subject or a portion thereof, wherein the imaging technique is capable of detecting one or more signals from the composition.

Statement 35: A method of treating an infectious disease in a subject in need thereof, the method comprising: administering to the subject a pharmaceutically effective amount of a composition according to any one of Statements 1-30.

Statement 36: A method of treating or preventing Cytokine Storm or Cytokine Release Syndrome in a subject having an infectious disease, the method comprising: administering to the subject a pharmaceutically effective amount of a composition according to any one of Statements 1-30.

Statement 37: The method according to Statement 36, further comprising: performing an imaging technique on the subject or a portion thereof, wherein the imaging technique is capable of detecting one or more signals from the composition; and making at least one treatment decision based on the results of the imaging technique performed on the subject.

Statement 38: A method of imaging a subject having an infectious disease in a subject, the method comprising: administering to the subject a pharmaceutically effective amount of a composition according to any one of Statements 1-30; and performing an imaging technique on the subject or a portion thereof, wherein the imaging technique is capable of detecting one or more signals from the composition.

Statement 39: A method of imaging a plurality of cells in a subject wherein the plurality of cells are infected with an infectious disease pathogen, the method comprising: administering to the subject a pharmaceutically effective amount of a composition according to any one of Statements 1-30 in a manner such that the plurality of cells effectively receive the composition; and performing an imaging technique on at least a portion of the subject containing the plurality of cells, wherein the imaging technique is capable of detecting one or more signals from the composition.

Statement 40: A method according to any one of Statements 31-39, wherein the imaging technique is selected from the group consisting of positron emission tomography (PET), computed tomography (CT), single photon emission computed tomography (SPECT), magnetic resonance imaging (MM), near-infrared (NIR), optical imaging, optoacoustic imaging, ultrasound, and any combination thereof.

Statement 41: A method according to any one of Statements 31-40, wherein the infectious disease is a viral infection.

Statement 42: A method according to any one of Statements 31-41, wherein the infectious disease is a respiratory viral infection selected from the group consisting of human influenza, the common cold, Middle East respiratory syndrome (MERS), severe acute respiratory syndrome coronavirus (SARS), and COVID-19.

Statement 43: A method according to any one of Statements 31-41, wherein the infectious disease is caused by infection by a virus selected from the group consisting of severe acute respiratory syndrome coronavirus (SARS-CoV), severe acute respiratory syndrome coronavirus 2 (SARS-CoV-2), Middle East respiratory syndrome-related coronavirus (MERS-CoV), human coronavirus NL63 (HCoV NL63), human coronavirus 0C43 (HCoV-0C43), human coronavirus HKU1 (HCoV HKU1), and human coronavirus 229E (HCoV-229E).

Statement 44: The composition according to any one of Statements 2-30, wherein the label is a non-radioactive metal capable of providing an advantageous therapeutic response at a tissue in the subject targeted by the conjugate.

Statement 45: The composition according to Statement 44, wherein the non-radioactive metal is selected from the group consisting of rhenium, platinum, copper, iron, arsenic, lead, tantalum, and any combination thereof.

Statement 46: A method according to any one of Statements 1-43, wherein the label is a radiotherapeutic label capable of delivering radiation to a tissue in the subject targeted by the conjugate.

Statement 47: The method according to Statement 46, wherein the label is a radiotherapeutic label capable of delivering radiation to an infected tissue in the subject and the advantageous therapeutic effect is increased viral particle inactivation, reduced viral replication, and/or reduced viral load in the tissue.

Statement 48: The method according to Statement 47, wherein the radiotherapeutic label is selected from the group consisting of Technetium-99, Gallium-68, Copper-60, Copper-64, Indium-111, Holmium-166, Rhenium-186, Rhenium-188, Yttrium-90, Lutetium-177, Radium-223, Actinium-225, and any combination thereof.

Statement 49: A method of treating an infectious disease and/or treating or preventing Cytokine Storm or Cytokine Release Syndrome in a subject in need thereof, the method comprising: administering to the subject a pharmaceutically effective amount of a composition according to any one of Statements 1-30 and 44-45; administering to the subject a therapeutically effective amount of a radiotherapeutic regimen; wherein the radiotherapeutic regimen delivers radiation to an infected tissue in the subject in need of treatment.

Statement 50: The method according to Statement 49, further comprising: administering to the subject a therapeutically effective amount of an antiviral compound and/or an anti-inflammatory compound.

Statement 51: The method according to Statement 50, wherein the antiviral compound is selected from the group consisting of remdesivir, oseltamivir phosphate, zanamivir, peramivir, baloxavir marboxil, darunavir, atazanavir, ritonavir, acyclovir, valacyclovir, valganciclovir, tenofovir, raltegravir, viral attachment inhibitors, viral entry inhibitors, uncoating inhibitors, protease inhibitors, polymerase inhibitors, nucleoside and nucleotide reverse transcriptase inhibitors, nonnucleoside reverse-transcriptase inhibitors, integrase inhibitors, and any combination thereof.

Statement 52: The method according to Statement 50 or Statement 51, wherein the anti-inflammatory compound may be selected from the group consisting of nonsteroidal anti-inflammatory drugs (NSAIDs), aspirin, ibuprofen, naproxen, corticosteroids, cyclooxygenase-2 (cox-2) inhibitors, salicylates, diclofenac, diflunisal, etodolac, celecoxib, etoricoxib, famotidine, flurbiprofen, indomethacin, ketoprofen, mefenamic acid, meloxicam, nabumetone, oxaprozin, piroxicam, sulindac, glucocorticoids, prednisone, cortisone, hydrocortisone, bethamethasone, prednisolone, triamcinolone, methylprednisolone, dexamethasone, ethamethasoneb, and any combination thereof.

Statement 53: The method according to any one of Statements 49-52, wherein the radiotherapeutic regimen causes increased viral particle inactivation, reduced viral replication, and/or reduced viral load in the tissue.

Statement 54: The method according to any one of Statements 49-53, wherein the radiotherapeutic regimen comprises administration of a radioactive agent selected from the group consisting of Technetium-99, Gallium-68, Copper-60, Copper-64, Indium-111, Holmium-166, Rhenium-186, Rhenium-188, Yttrium-90, Lutetium-177, Radium-223, Actinium-225, and any combination thereof.

Statement 55: The method according to Statement 54, further comprising: performing an imaging technique on the subject or a portion thereof, wherein the imaging technique is capable of detecting one or more signals from the composition; and making at least one treatment decision based on the results of the imaging technique performed on the subject.

Statement 56: A method of treating an infectious disease and/or treating or preventing Cytokine Storm or Cytokine Release Syndrome in a subject in need thereof, the method comprising: administering to the subject a pharmaceutically effective amount of the combination therapy composition according to any one of Statements 2-30 and 44-45; wherein the label provides an advantageous therapeutic effect in treating the infectious disease.

Statement 57: The method according to Statement 56, wherein the label is a radiotherapeutic label capable of delivering radiation to an infected tissue in the subject and the advantageous therapeutic effect is decreased inflammation, increased viral particle inactivation, reduced viral replication, and/or reduced viral load in the tissue.

Statement 58: The method according to Statement 57, wherein the label is selected from the group consisting of Technetium-99, Gallium-68, Copper-60, Copper-64, Indium-111, Holmium-166, Rhenium-186, Rhenium-188, Yttrium-90, Lutetium-177, Radium-223, Actinium-225, and any combination thereof.

Statement 59: The method according to Statement 56, wherein the label is a non-radioactive metal that is toxic to an infectious diseases causing agent and the advantageous therapeutic effect is decreased inflammation, toxicity-induced cell death or viral particle inactivation, reduced viral replication, and/or reduced viral load in the tissue.

Statement 60: The method according to Statement 59, wherein the non-radioactive metal is selected from the group consisting of rhenium, platinum, copper, iron, arsenic, lead, tantalum, and any combination thereof.

Statement 61: The method according to any one of Statements 46-60, wherein the infectious disease is a viral infection.

Statement 62: The method according to any one of Statements 46-60, wherein the infectious disease is a respiratory viral infection selected from the group consisting of human influenza, the common cold, Middle East respiratory syndrome (MERS), severe acute respiratory syndrome coronavirus (SARS), and COVID-19.

Statement 63: The method according to any one of Statements 46-60, wherein the infectious disease is caused by infection by a virus selected from the group consisting of severe acute respiratory syndrome coronavirus (SARS-CoV), severe acute respiratory syndrome coronavirus 2 (SARS-CoV-2), Middle East respiratory syndrome-related coronavirus (MERS-CoV), human coronavirus NL63 (HCoV NL63), human coronavirus 0C43 (HCoV-0C43), human coronavirus HKU1 (HCoV HKU1), and human coronavirus 229E (HCoV-229E).

Statement 64: The method according to any one of Statements 46-63, wherein the imaging technique is selected from the group consisting of positron emission tomography (PET), computed tomography (CT), single photon emission computed tomography (SPECT), magnetic resonance imaging (MM), near-infrared (NIR), optical imaging, optoacoustic imaging, ultrasound, and any combination thereof.

What is claimed is:

1. A method of imaging a plurality of cells in a subject wherein the plurality of cells are infected with an infectious disease pathogen, the method comprising:
administering to the subject a pharmaceutically effective amount of a composition in a manner such that the plurality of cells effectively receive the composition, the composition comprising: a conjugate of a nucleoside analog, a chelator, and a label; and
performing an imaging technique on at least a portion of the subject containing the plurality of cells, wherein the imaging technique is capable of detecting one or more signals from the composition.

2. The method according to claim 1, wherein the nucleoside analog is selected from the group consisting of adenine, adenosine, deoxyadenosine, guanine, guanosine, dexoyguanosine, thymine, 5-methyluridine, thymidine, uracile, uridine, deoxyuridine, cytosine, cytidine, deoxycytidine, and any combination thereof

3. The method according to claim 2, wherein the chelator is selected from the group consisting of an aminated chelator, an acid chelator, a cyclam, a N4 chelator or ligand, 6-carboxy-1,4,8,11-tetraazaundecane, and 1,4,8,11-tetraazabicyclohexadecane.

4. The method according to claim 3, wherein the label is selected from the group consisting of Technetium-99, Gallium-68, Copper-60, Copper-64, Indium-111, Holmium-166, Rhenium-186, Rhenium-188, Yttrium-90, Lutetium-177, Radium-223, Actinium-225, and any combination thereof.

5. The method according to claim 1, wherein the conjugate comprises a conjugate compound having a structure according to Formula I:

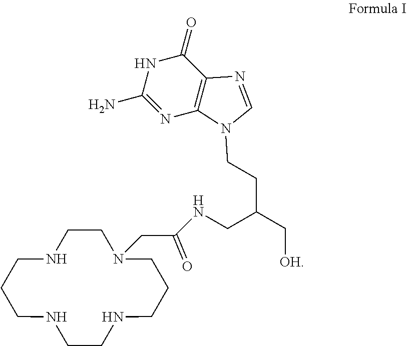 [SMILES: Nc1nc2c(ncn2CCC(CO)CNC(=O)CN2CCCNCCNCCCNCC2)c(=O)[nH]1]

6. The method according to claim 1, wherein the conjugate comprises a conjugate compound having a structure according to Formula II:

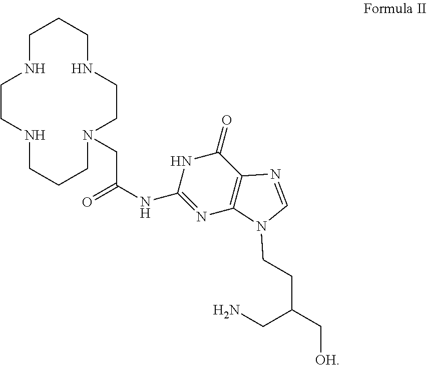 [SMILES: NCC(CO)CCn1cnc2c(=O)[nH]c(NC(=O)CN3CCCNCCNCCCNCC3)nc21]

7. The method according to claim 1, wherein the conjugate comprises a conjugate compound having a structure according to Formula III:

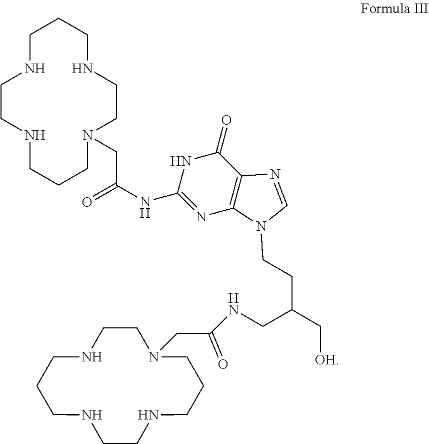 [SMILES: O=C(CN1CCCNCCNCCCNCC1)NCC(CO)CCn1cnc2c(=O)[nH]c(NC(=O)CN3CCCNCCNCCCNCC3)nc21]

8. The method according to claim 1, wherein the conjugate comprises a conjugate compound having a structure according to Formula IV:

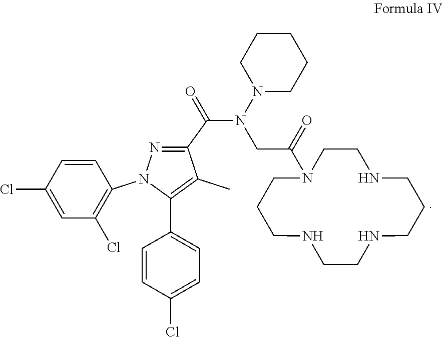 [SMILES: Cc1c(C(=O)N(CC(=O)N2CCCNCCNCCCNCC2)N2CCCCC2)nn(-c2ccc(Cl)cc2Cl)c1-c1ccc(Cl)cc1]

9. The method according to claim 1, wherein the imaging technique is selected from the group consisting of positron emission tomography (PET), computed tomography (CT), single photon emission computed tomography (SPECT), magnetic resonance imaging (MRI), near-infrared (NIR), optical imaging, optoacoustic imaging, ultrasound, and any combination thereof

10. A method according to claim 1, wherein the infectious disease pathogen is a virus selected from the group consisting of severe acute respiratory syndrome coronavirus (SARS-CoV), severe acute respiratory syndrome coronavirus 2 (SARS-CoV-2), Middle East respiratory syndrome-related coronavirus (MERS-CoV), human coronavirus NL63 (HCoV NL63), human coronavirus OC43 (HCoV-OC43), human coronavirus HKU1 (HCoV HKU1), and human coronavirus 229E (HCoV-229E).

11. The method according to claim 1, wherein the label is a non-radioactive metal capable of providing an advantageous therapeutic response at a tissue in the subject targeted by the conjugate, wherein the advantageous therapeutic effect is increased viral particle inactivation, reduced viral replication, and/or reduced viral load in the tissue.

12. The method according to claim 11, wherein the non-radioactive metal is selected from the group consisting of rhenium, platinum, copper, iron, arsenic, lead, tantalum, and any combination thereof.

13. The method according to claim 1, wherein the label is a radiotherapeutic label capable of delivering radiation to a tissue in the subject targeted by the conjugate.

14. The method according to claim 13, wherein the radiotherapeutic label is selected from the group consisting of Technetium-99, Gallium-68, Copper-60, Copper-64, Indium-111, Holmium-166, Rhenium-186, Rhenium-188, Yttrium-90, Lutetium-177, Radium-223, Actinium-225, and any combination thereof.

15. The method according to claim 14, further comprising:
administering to the subject a therapeutically effective amount of an antiviral compound and/or an anti-inflammatory compound.

16. The method according to claim 15, wherein the antiviral compound is selected from the group consisting of remdesivir, oseltamivir phosphate, zanamivir, peramivir, baloxavir marboxil, darunavir, atazanavir, ritonavir, acyclovir, valacyclovir, valganciclovir, tenofovir, raltegravir, viral attachment inhibitors, viral entry inhibitors, uncoating inhibitors, protease inhibitors, polymerase inhibitors, nucleoside and nucleotide reverse transcriptase inhibitors, nonnucleoside reverse-transcriptase inhibitors, integrase inhibitors, and any combination thereof.

17. The method according to claim 16, wherein the anti-inflammatory compound may be selected from the group consisting of nonsteroidal anti-inflammatory drugs (NSAIDs), aspirin, ibuprofen, naproxen, corticosteroids, cyclooxygenase-2 (cox-2) inhibitors, salicylates, diclofenac, diflunisal, etodolac, celecoxib, etoricoxib, famotidine, flurbiprofen, indomethacin, ketoprofen, mefenamic acid, meloxicam, nabumetone, oxaprozin, piroxicam, sulindac, glucocorticoids, prednisone, cortisone, hydrocortisone, bethamethasone, prednisolone, triamcinolone, methylprednisolone, dexamethasone, ethamethasoneb, and any combination thereof.

18. The method according to claim 9, further comprising:
making at least one treatment decision based on the results of the imaging technique performed on the subject.

19. The method according to claim 1, wherein the label is a radiotherapeutic label capable of delivering radiation to an infected tissue in the subject and the advantageous therapeutic effect is decreased inflammation, increased viral particle inactivation, reduced viral replication, and/or reduced viral load in the tissue.

20. The method according to claim 1, wherein the label is a non-radioactive metal that is toxic to an infectious diseases causing agent and the advantageous therapeutic effect is decreased inflammation, toxicity-induced cell death or viral particle inactivation, reduced viral replication, and/or reduced viral load in the tissue, wherein the non-radioactive metal is selected from the group consisting of rhenium, platinum, copper, iron, arsenic, lead, tantalum, and any combination thereof.
